(19) United States
(12) Patent Application Publication
(10) Pub. No.: US 2011/0287413 A1
(43) Pub. Date: Nov. 24, 2011

(54) MICROFLUIDIC SYSTEM

(75) Inventors: Julien Chapron, Midlothian (GB); Lidia Prieto-Lafuente, Midlothian (GB)

(73) Assignee: ITI SCOTLAND LIMITED, Glasgow, GB (GB)

(21) Appl. No.: 13/060,045

(22) PCT Filed: Aug. 12, 2009
(86) PCT No.: PCT/EP2009/060460

§ 371 Date: Aug. 10, 2011

(30) Foreign Application Priority Data
Aug. 22, 2008 (GB) 0815472.6

(51) Int. Cl. C12Q 1/68; C12Q 1/70; C12M 1/34; G01N 33/53; G01N 33/58; G01N 31/10; G01N 33/92; G01N 33/68; G01N 33/50; B01J 8/00; G01N 31/00

(52) U.S. Cl. 435/6.1; 422/430; 422/603; 435/5; 435/7.1; 435/287.1; 435/287.2; 436/501; 436/8; 436/37; 436/69; 436/71; 436/86; 436/87

(57) ABSTRACT

A microfluidic system comprising a 1^{st} reaction zone, a 2^{nd} reaction zone, a reagent delivery channel configured to deliver one or more reagents to the 1^{st} reaction zone, a waste channel to remove waste from the 2^{nd} reaction zone, a 1^{st} sample delivery channel configured to deliver a sample to the 1^{st} reaction zone and a 2^{nd} sample delivery channel configured to deliver a sample to the 2^{nd} reaction zone; wherein the microfluidic system comprises a means for retaining one or more reagents in each reaction zone; and wherein the 1^{st} reaction zone and 2^{nd} reaction zone are connected in series by a reaction zone channel.

The present invention relates to a microfluidic system, more specifically to a microfluidic system for assaying a sample, especially a biological sample. The microfluidic system is configured to allow two samples, such as a test sample and a control, to be processed under the same reaction conditions without cross contamination. The invention also concerns a cartridge system comprising the microfluidic system, and assays performed using the microfluidic system or cartridge system.

Microfluidics relates to the manipulations of fluids that are constrained in microscale. Microfluidic systems have been used in many different fields which require the use of very small volumes of fluids, including engineering and biotechnology. For example, microfluidic systems have been used in the development of inkjet printheads and DNA chips.

It is known to employ microfluidic systems in biological assays. Microfluidic biochips allow assay operations such as detection, sample pre-treatment and sample preparation on one chip. An emerging application area for biochips is clinical pathology, particularly in immediate point-of-care diagnosis.

The development of microfluidic processing devices and chips has facilitated the development of cartridges used for biological assays, since microfluidics allows much smaller (and cheaper) cartridges to be produced which can readily be inserted into a larger robust assay device. Published international application WO 02/090995 describes one such cartridge, which may be employed in a near-patient environment assay process. PCT/GB07/003,666 also describes a cartridge system for use in a biological assay, wherein microfluidics may be employed.

In any assay method, it is necessary to compare the results to a standard curve and/or a control. Comparison of the results from the test sample to a control allows any background data, which is does not result from the test sample, to be taken into consideration. Comparison of the results from the test sample to a standard curve allows calculation of the quantity of an analyte in the test sample. It is well known to include a control chamber and an experiment chamber for the test sample in a microfluidic assay system. It is necessary for the control chamber and the experiment chamber to be kept separate to prevent cross contamination. However, it is also highly desirable to ensure that both chambers are subjected to the same reaction conditions and pass through similar channels/conduits. In many microfluidic systems this is achieved by setting the control and the experiment chambers in parallel. The system is configured to split the reagents before entry into each chamber set in parallel.

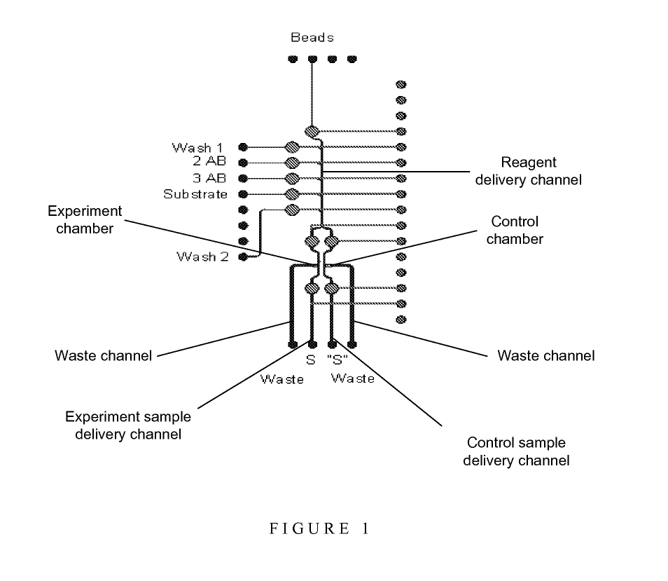

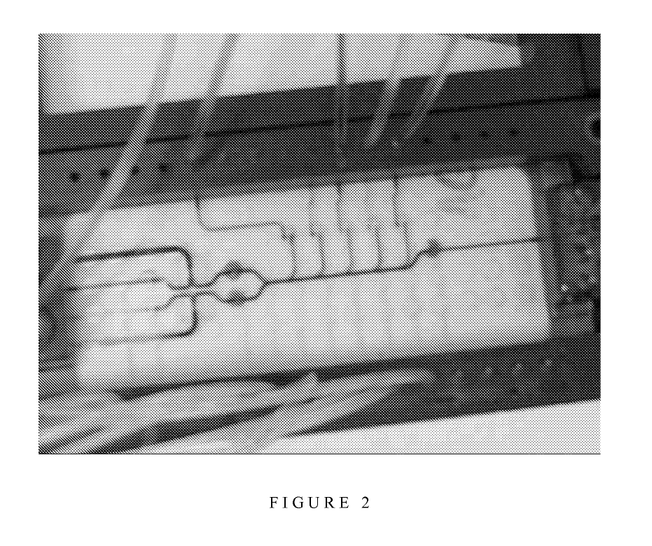

An example of such a parallel system is shown in FIG. 1 and FIG. 2. In FIG. 1, the reagents are conveyed from the top of the system through a reagent delivery channel. The reagent delivery channel splits and conveys the reagents separately into the control chamber and the experiment chamber. The control sample and experiment sample are conveyed separately to the chambers in order to avoid cross contamination of the experiment sample into the control chamber. The experiment sample is conveyed to the experiment chamber through an experiment sample delivery channel and then is delivered into a waste channel (left side of system). In the same way, a control, such as a buffer, is conveyed to the control chamber through a separate control sample delivery channel and then delivered into a separate waste channel (right side of system).

This so-called “parallel system” is designed to allow both chambers to receive the same amount of reagents and prevent cross contamination of the experiment sample with the control. However, accurate splitting of the reagents in microfluidic systems is difficult and in practice most of the reagents flow into one chamber and is not evenly distributed. In FIG. 2, it can be seen that the reagent solution (shown by dark lines in the channels) has filled the top channel and chamber after the split, whereas the bottom channel and chamber contains little reagent solution (as shown by the lighter coloured channels). This is a significant problem because if the reagents are not equally distributed between the chambers, the two chambers are not subjected to the same quantity of reagents and the results of the assay from each chamber are not comparable.

The unequal distribution of reagent solution after the split in the channel is due to the kinetics of fluids at the microscale. Fluids behave differently at the microscale compared to the macroscale. This is because factors such as surface tension, energy dissipation and fluidic resistance affect the flow of the fluids to a greater extent at the microscale. One of these effects is that a fluid at the microscale will not easily divide equally between two channels, the system inherently wants to remain a steady laminar flow.

Further developments in microfluidic systems are still required to improve their application to assay methods. In particular, there is a need for the development of new microfluidic systems which overcome the problems associated with known microfluidic systems, such as those described above.

Accordingly, the present invention provides a microfluidic system comprising a 1^{st} reaction zone, a 2^{nd} reaction zone, a reagent delivery channel configured to deliver one or more reagents to the 1^{st} reaction zone, a waste channel to remove waste from the 2^{nd} reaction zone, a 1^{st} sample delivery channel configured to deliver a sample to the 1^{st} reaction zone and a 2^{nd} sample delivery channel configured to deliver a sample to the 2^{nd} reaction zone; wherein the microfluidic system comprises a means for retaining one or more reagents in each reaction zone; and wherein the 1^{st} reaction zone and 2^{nd} reaction zone are connected in series by a reaction zone channel.

The two reaction zones are set in series thus allowing the same amount of one or more reagents to be entered into each reaction chamber. In contrast to the parallel system, the present invention does not split the solution comprising the reagents into two separate channels and, therefore, avoids the problem of the unequal distribution of the reagents in the two reaction zones which occurs in the parallel system. The present invention allows two samples to be analysed under sufficiently similar reaction conditions to produce assay results which are sufficiency accurate for comparison of the two samples, preferably by calibrating one sample with the other sample. For example, the results obtained using the system according to the present invention in an assay method allow more accurate comparison of a test sample with a calibration sample. The reaction conditions in the two reaction zones are preferably substantially identical, more preferably the reaction conditions are identical.

The system according to the present invention is also easier and more cost effective to manufacture due to the simplified design.

The present invention also provides a cartridge system comprising:
(a) a reagent component for storing one or more reagents; and
(b) a processing component for processing the one or more reagents in an assay,
   wherein the processing component comprises a microfluidic system according as defined above;
   wherein the reagent component and the processing component are configured to be coupled together to form a cartridge.

The present invention also provides the use of a microfluidic system as defined in above or a cartridge system as defined above in an assay method for identifying an analyte in a sample.

The present invention also provides an assay method for one or more analytes in a sample, which method comprises:
a) conveying a solution comprising one or more reagents through a microfluidic system as defined above, wherein the solution comprising one or more reagents is conveyed into the 1^{st} reaction zone and the 2^{nd} reaction zone;
b) retaining one or more reagents in the 1^{st} reaction zone and 2^{nd} reaction zone;
c) conveying a sample through the 1^{st} sample delivery channel into the 1^{st} reaction zone and conveying a sample through the 2^{nd} sample delivery channel into the 2^{nd} reaction zone; and
d) assaying for the one or more analytes.

The present invention will now be described in detail. To aid in this description, reference is made by way of example only to the following Figures, in which:

FIGS. 1 and 2 show the parallel microfluidic system known in the art.

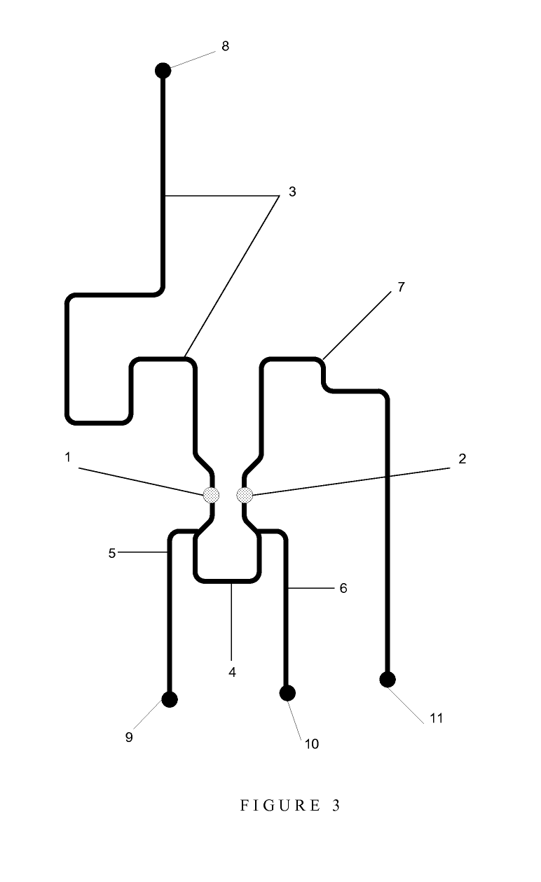

FIG. 3 illustrates a microfluidic system according to the present invention—1 is the 1^{st} reaction zone, 2 is the 2^{nd} reaction zone, 3 is the reagent delivery channel, 4 is the reaction zone channel, 5 is the 1^{st} sample delivery channel, 6 is the 2^{nd} sample delivery channel, 7 is the waste channel from the 2^{nd} reaction zone, 8 is the inlet for one or more reagents, 9 is the inlet for the sample for the 1^{st} reaction zone, 10 is the inlet for the sample for the 2^{nd} reaction zone and 11 is an outlet for the reagents and the sample from the 2^{nd} reaction zone.

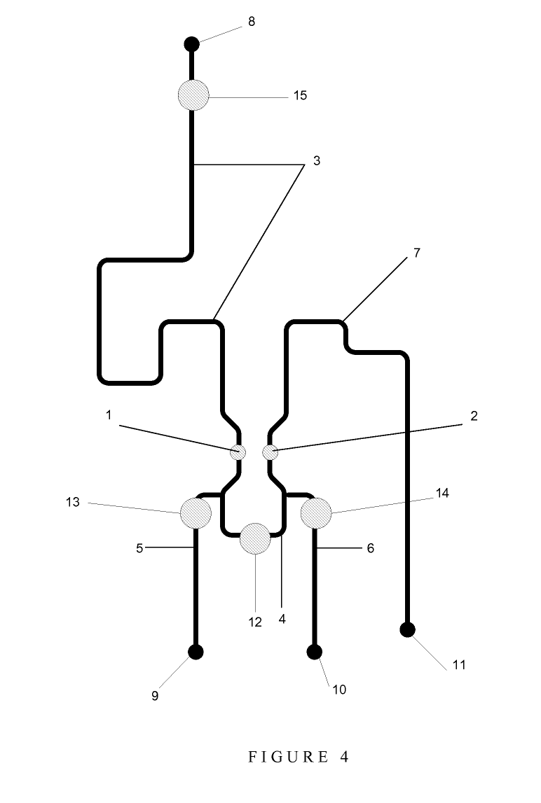

FIG. 4 shows preferred features of the microfluidic system, wherein 12 is a reaction zone valve and 13 and 14 are valves in the sample delivery channels.

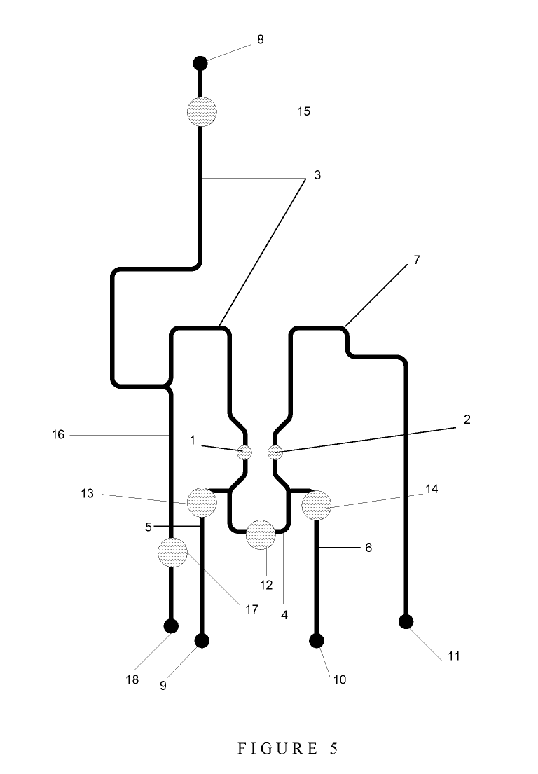

FIG. 5 shows preferred features of the microfluidic system, wherein 15 is a valve in the reagent delivery channel, 16 is a further waste channel, 17 is a valve in the further waste channel and 18 is an outlet from the further waste channel.

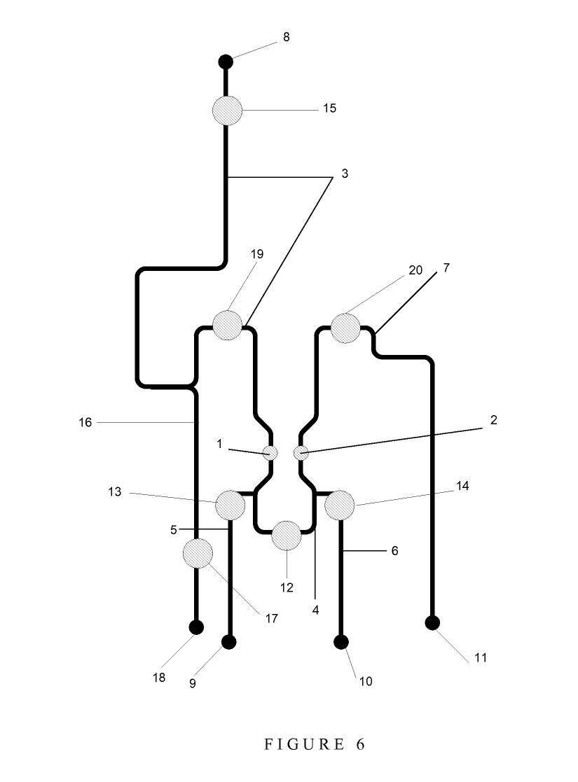

FIG. 6 shows preferred features of the microfluidic system, wherein 19 is a further valve in the reagent delivery channel positioned after the bifurcating region and 20 is a valve in the waste channel from the 2^{nd} reaction zone.

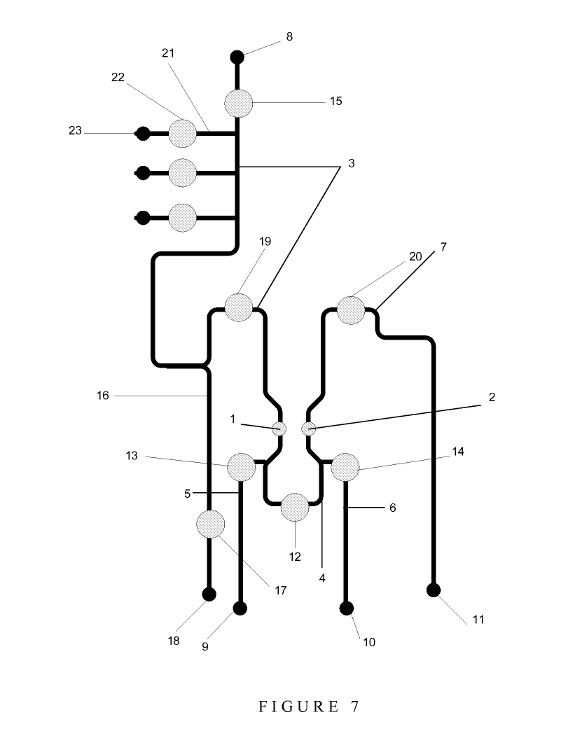

FIG. 7 shows preferred features of the microfluidic system, wherein 21 is a delivery channel for delivering a fluid to the reagent delivery channel, 22 is a valve in the delivery channel and 23 is an inlet for the delivery channel.

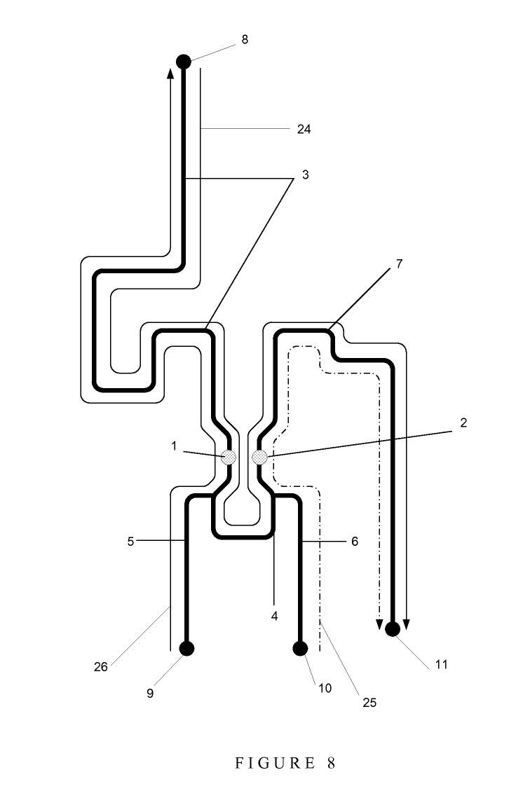

FIG. 8 shows an embodiment of the present invention wherein 24 represents the flow of the one or more reagents through the reagent delivery channel into the 1^{st} reaction zone, through the reaction zone channel into the 2^{nd} reaction zone and through the waste channel, 25 represents the flow of the sample through the 2^{nd} sample delivery channel to the 2^{nd} reaction zone and through the waste channel and 26 shows the flow of the sample through the 1st sample delivery channel to the 1^{st} reaction zone and through the reagent delivery channel.

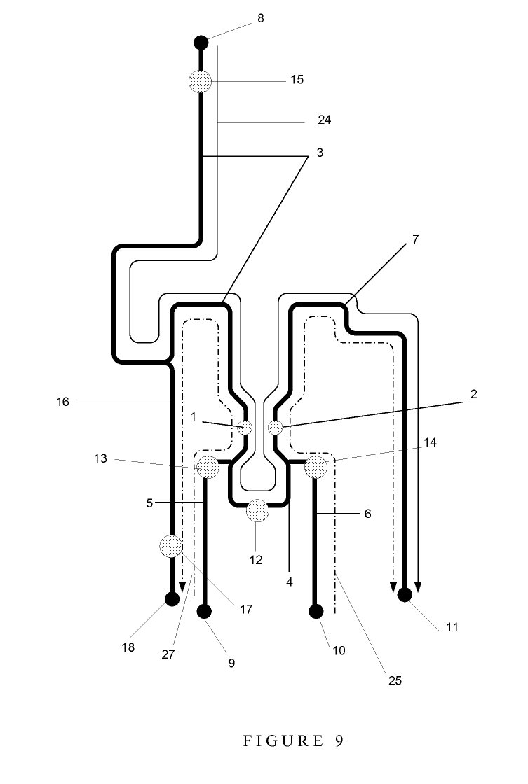

FIG. 9 shows an embodiment of the present invention wherein 24 represents the flow of the one or more reagents through the reagent delivery channel into the 1^{st} reaction zone, through the reaction zone channel into the 2^{nd} reaction zone and through the waste channel, 25 represents the flow of the sample through the 2^{nd} sample delivery channel to the 2^{nd} reaction zone and through the waste channel and 27 shows the flow of the sample through the 1^{st} sample delivery channel to the 1^{st} reaction zone and through the further waste channel.

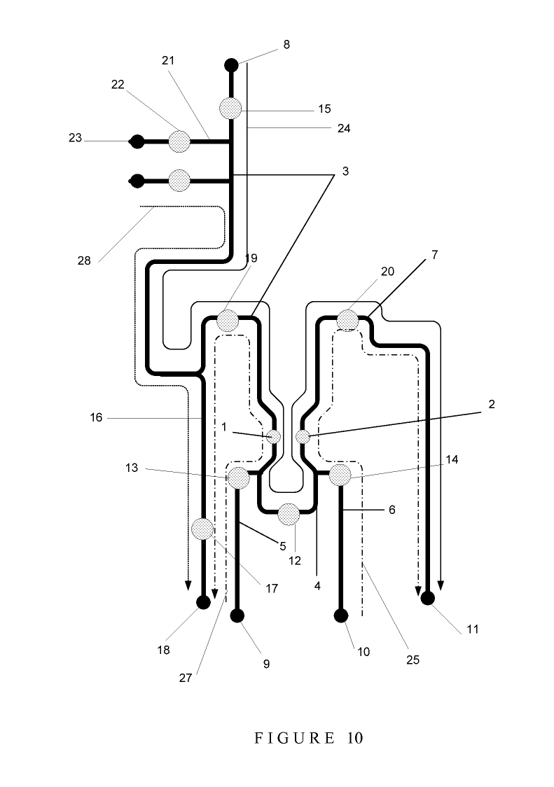

FIG. 10 shows the flow of reagents 24 and flow of samples 25 and 27 as for FIG. 9 and further shows 28 which represents the flow of a fluid from a delivery channel into the reagent delivery channel and through the further waste channel without entering the 1^{st} reaction zone or the 2^{nd} reaction zone.

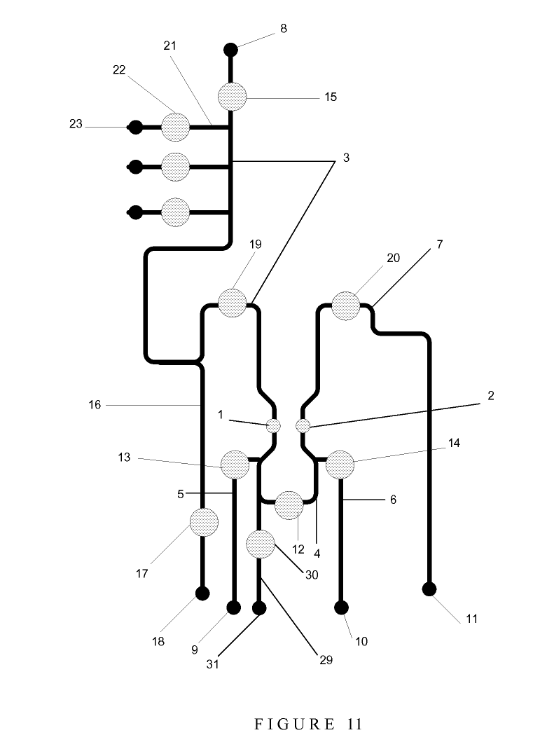

FIG. 11 shows preferred features of the microfluidic system, wherein 29 is a reaction zone waste channel positioned in the reaction zone channel, 30 is a valve in the reaction zone waste channel and 31 is an outlet from the reaction zone waste channel.

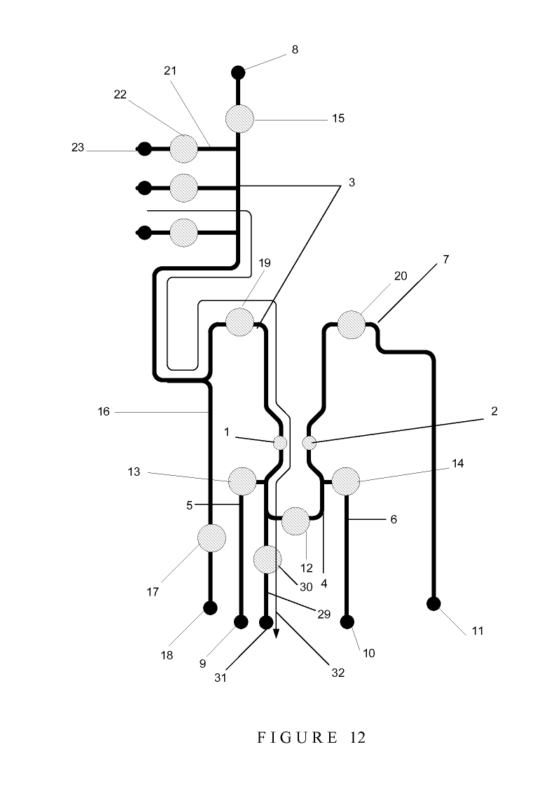

FIG. 12 shows the flow of a fluid from a delivery channel into the reagent delivery channel and the 1^{st} reaction zone and through the reaction zone waste channel in the reaction zone channel without entering the second reaction zone.

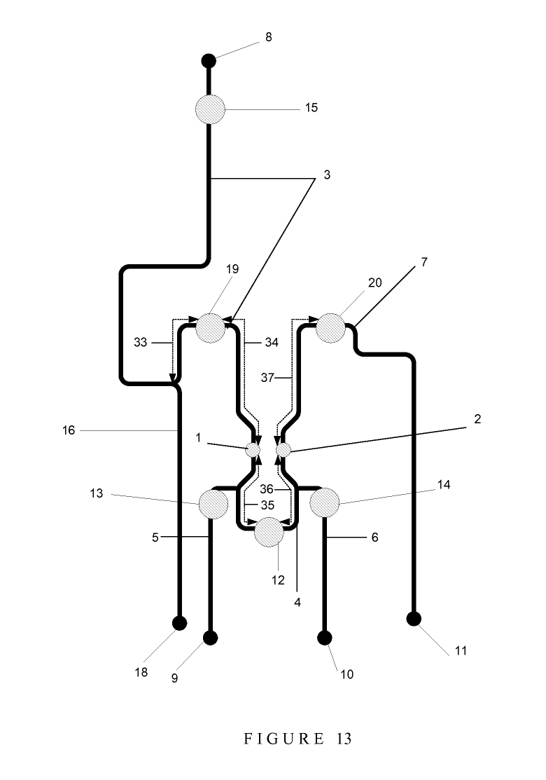

FIG. 13 shows a microfluidic system where different regions of the reagent delivery channel 33 and 24, the reaction zone channel 35 and 36 and the waste channel from the 2^{nd} reaction zone 37 are shown. In a preferred embodiment the total volume of fluid required to fill regions 33 and 34 is the same as the total volume of fluid required to fill regions 35 and 35. In a further preferred embodiment the total volume of fluid required to fill regions 34 and 35 is the same as the total volume of fluid required to fill regions 36 and 37. In a further preferred embodiment, the same volume of fluid is required to fill each of regions 33, 34, 35, 36 and 37,

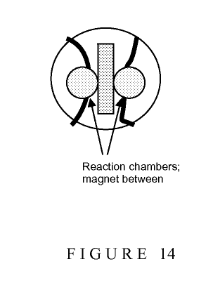

FIG. 14 shows a zoomed in section of the reaction zones of the microfluidic system according to the present invention where the means for retaining one or more reagents is a magnet positioned between the two reaction chambers.

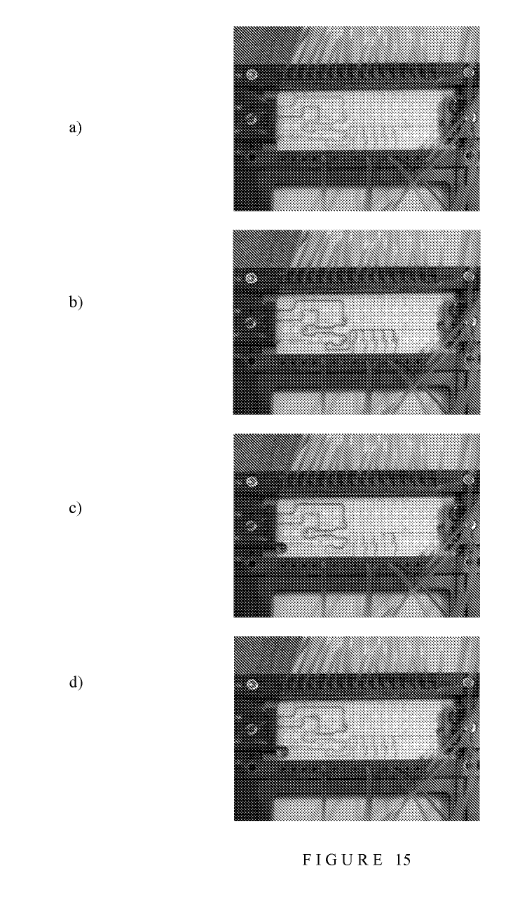

FIGS. 15a to 15d shows a microfluidic system according to the present invention at different stages of an assay, wherein FIG. 15a shows the application of surface treatment solution (i.e. BSA); FIG. 15 b shows the system when a reagent solution comprising beads has been conveyed through the reagent delivery channel into the 1^{st} reaction zone, through the reaction zone channel into the 2^{nd} reaction zone and through the waste channel; FIG. 15 c shows the system when a wash solution has been conveyed through the reagent delivery channel and the waste channel (from the 1^{st} reaction zone) without passing through the reaction zones to clear reagent delivery channel and waste channel of bead solution; and FIG. 15 d shows the system when reagents have been conveyed to the reaction zones and one sample is conveyed through a sample delivery channel to the reaction zone.

The microfluidic system according to the present invention comprises a plurality of interconnecting channels and two reaction zones. Microfluidic systems suitable for transporting, mixing, incubating and examining liquids are known and, therefore, the skilled person would be able to manufacture a system according to the present invention using common general knowledge. Fluids are entered into the channels of the system through inlet ports and removed through exit ports. The system may comprise a microfluidic pumping means which are well known in the art.

The 1^{st} reaction zone and 2^{nd} reaction zone may be any means suitable for allowing a sample to react with one or more reagents. The reaction zones may be in the form of a chamber or a region of the microfluidic channel.

The system of the present invention will typically be employed in an assay for identifying an analyte in a sample. The present invention also provides an assay method for one or more samples in a sample.

In such assays, it is the norm to test a sample from a patient (test sample) in order to establish a diagnosis (sometimes in combination with a preferred treatment—termed theranostics). The sample will be assayed with a view to detecting the identity and/or quantity of a particular analyte which may be in the sample. The type of analyte is not especially limited, and the system of the invention may be adapted to many types of analyte, including assays for multiple analytes, sequentially or simultaneously. Typically, the analyte is selected from a biological molecule, a pathogen, a virus or virus component, and a cell or a cell component. Examples of analytes include whole cells such as liver cells, enzymes, whole viruses (e.g. Hepatitis C virus (HCV) and Human Immunodeficiency Virus (HIV)), bacterial pathogens (E coli or S aureus), proteins polypeptides and peptides, and nucleic acids such as DNA and/or RNA. Also included are carbohydrates and small molecules, such as drugs, pharmaceuticals and metabolites.

The samples entered into the 1^{st} reaction zone and 2^{nd} reaction zone may both be test samples, for example samples taken from a patient. The test samples are typically selected from blood, saliva, urine, amniotic fluid, mucus, ascites fluid, pulmonary liquids (including pleural), lavage (for example pulmonary eastric etc.), biopsy fluid, semen, swabs (for example PAP, oral etc.), sweat, tears, faecal stools, cerebrospinal fluid, wound exudate, synovial fluid and the like. The samples may be taken from a single patient or from different patients.

In an alternative embodiment, one sample is a test sample and the other sample is a calibration sample comprising one or more aliquots of an analyte, each aliquot having a different known quantity of the one or more analytes. The quantity of the analyte in each aliquot may be selected to reflect the known or suspected variation in the quantity of the analyte in the test sample. The quantity of the analyte may indicate the presence and/or a stage of a particular disease or the efficacy and/or toxicity of a therapy.

In this embodiment, wherein one sample is a test sample and the other sample is a calibration sample comprising one or more aliquots of an analyte, the assay method according to the present invention preferably comprises a step of calibrating the quantity of the one or more analytes in the test sample against the known and determined quantity of the one or more analytes in the calibration sample.

Alternatively, one sample is a test sample and the other sample is a calibration sample comprising a control liquid. The control liquid may be any suitable blank or standard sample which may be required in the assay method. The control liquid may, for examples, comprise the same reagents that the test sample is mixed with.

In this embodiment, wherein one sample is a test sample and the other sample is a calibration sample comprising a control liquid, the assay method according to the present invention preferably comprises a step of calibrating the assay results of the reaction zone comprising the test sample with the assay results of the reaction zone comprising the calibration sample. For example, where the sample conveyed into the 1^{st} reaction zone is a test sample and the sample conveyed into the 2^{nd} reaction zone is a calibration sample, the assay results from the 1^{st} reaction zone is calibrated with the assay results of the 2^{nd} reaction zone.

The one or more reagents may be any suitable reagent as required for a selected assay. The one or more reagents are typically independently selected from a protein, a polypeptide, a peptidomimetic, a nucleic acid, an oligonucleotide, an aptamer and organic or inorganic chemical reagents. Preferably the one or more reagents are independently selected from an antibody or a fragment of an antibody, a receptor or a fragment of a receptor, an antigen, an enzyme, an enzyme inhibitor, a binding protein, a catalyst, a blood clotting activator, an anticoagulant, a serum separating agent, a detergent and a salt.

The reagents are preferably for enzymatic assays, RNA and DNA assays, protein assays or ALT enzymatic assays. Examples of preferred reagents include protease inhibitors, RNAse inhibitors, RNAase, reverse transcriptase, DNA polymerase, SYBR green,=concanavalin A, magnetic antibodies, magnetic microparticles and magnetic nanoparticles such as MACS® beads (Miltenyi Biotech™), guanidine isothiocyanate, glycogen, RNA carrier, sodium chloride, dNTPs, magnesium chloride, and bridging agents such as StemSep®.

In one embodiment the one or more reagents are labels for one or more analytes. The analyte preferably comprises a pathogen, particularly a virus or virus particle or virus component, a protein, a polypeptide, a glycoprotein, a nucleic acid, such as DNA or RNA, an oligonucleotide, a metabolite, a carbohydrate such as a complex carbohydrate, a lipid, a fat, or an endogeneous or exogeneous small molecule such as a pharmaceutical or drug.

In one embodiment the one or more reagents are magnetic or magnetisable and/or are attached to a magnetic or magnetisable substance. In this embodiment the one or more reagents may be magnetic beads or the one or more reagents are attached to magnetic beads.

Alternatively the one or more reagents may be magnetic proteins or the one or more reagents are attached to magnetic proteins. In one embodiment, the reagent may be a magnetic liposome or a similar biological entity.

In a preferred embodiment, when the one or more reagents are magnetic or magnetisable and/or are each attached to a magnetic or magnetisable substance, each of the one or more reagents comprise a label for analyte. Each reagent may comprise one or more of the same or different types of labels.

Preferably the one or more reagents each comprise one or more labels for one or more analytes, which label is attached to a magnetic or magnetisable substance, the label comprising:
(a) a recognition moiety for attaching the one or more labels to the one or more analytes; and
(b) a moiety for binding or encapsulating the magnetic or magnetisable substance;
   wherein the moiety for binding or encapsulating the magnetic or magnetisable substance comprises a metal-binding protein, polypeptide, or peptide.

PCT/GB07/004,188 (the contents of which are incorporated by reference) discloses the above magnetic recognition labels, which are capable of attaching small quantities of a magnetic (or magnetisable) substance to an analyte via a recognition agent for the analyte. The labels have significant advantages in that they are capable of attaching a very small volume of the magnetic substance to the analyte, so that the analyte can be influenced by magnetic fields, even in the confined space of the microfluidic system according to the present invention.

One or more reagents conveyed through the microfluidic reaction system are suitable for being retained in the reaction zones. For example, reagents which are magnetic or magnetisable and/or are attached to a magnetic or magnetisable substance are particularly suitable to be retained in the reaction zone by means of a magnet.

One or more further reagents may also be conveyed through the microfluidic system, which are not suitable for being retained in the reaction zones.

The means for retaining one or more reagents in the reaction zones is not particularly limited and may comprise any suitable physical, biological or chemical means. The means for retaining may be positioned in the reaction zones or the means for retaining may be positioned outside of the reaction zones if they do not require physical contact with the one or more reagents.

An example of a physical means for retaining is an adapted configuration of the reaction zones which enables one or more reagents to settle or be trapped within the reaction zones.

The means for retaining may be biological in nature, wherein the whole or part of the internal surface of the reaction zones are treated with an agent capable of forming a covalent or non-covalent bond with one or more reagents thereby capturing the one or more reagents in the reaction zones. An example of this would be an antibody or fragment thereof or a nucleic acid probe strand complementary to the target strand which has been attached to the reaction zone.

The means for retaining may be chemical in nature, wherein the whole or part of the internal surface of the reaction zones are treated with an agent capable of forming a covalent or non-covalent bond with one or more reagents thereby capturing the one or more reagents in the reaction zones. The means may be any suitable chemical entity which is activated by sequential reagent chemistries.

In a preferred embodiment the means for retaining the one or more reagents is magnetic or magnetisable and, therefore, capable of retaining reagents which are magnetic or magnetisable and/or are attached to a magnetic or magnetisable substance. In this embodiment, the means for retaining is preferably a magnet. In a preferred embodiment the means for retaining one or more reagents is positioned so that it affects each reaction zone equally. The configuration of the microfluidic system preferably allows the 1^{st} reaction zone and 2^{nd} reaction zone to be in close proximity to each other. Therefore, one magnet maybe positioned between the 1^{st} reaction zone and the 2^{nd} reaction zone, as shown for example in FIG. 14. This is particularly advantageous because it ensures that both reaction zones are subjected to the same magnetic field strength and, therefore, the same quantity of the reagents is retained in each reaction zone.

Alternatively, the means for retaining may be two magnets each positioned adjacent to a reaction chamber. The magnet may be an electromagnet.

The means for retaining the one or more reagents is preferably capable of being turned on and off and/or added and removed from the system. For example, in the embodiment wherein the means for retaining is a magnet, the magnet may be added and removed from the position between the two reaction zones. In the embodiment wherein the magnet is an electromagnet, the current supplying the electromagnet may simply be turned on and off In a preferred embodiment of the assay method according to the present invention, the means for retaining one or more reagents are turned on or added to the 1^{st} reaction zone and 2^{nd} reaction zone in step b) after the solution comprising the one or more reagents has filled the 1^{st} reaction zone and the 2^{nd} reaction zone. Preferably the flow of the solution has stopped when the means for retaining the one or more reagents are turned on or added. This embodiment is advantageous because if the one or more reagents are retained in the reaction zones whilst the solution comprising the reagents is still filling the system, the concentration of the reagents in the solution would change and a different concentration of reagents would enter the 1^{st} reaction zone and the 2^{nd} reaction zone. Accordingly, in this embodiment where the reagents are retained in the reaction zones when the solution comprising the reagents has filled the 1^{st} and 2^{nd} reaction zones and preferably the flow as stopped, the same concentration of reagents is present in each reaction zone when the means for retaining the one or more reagents is turned on or added. This ensures that the reaction conditions in both reaction zones are the same.

In one embodiment the microfluidic system according to the present invention does not contain valves to open and close the microfluidic channels. In this embodiment, the flow of the fluids through the channels may be controlled by configuration of the microfluidic channels. As discussed above, in the parallel system known in the art there is an unequal distribution of reagent solution after the split in the channel due to the kinetics of fluids at the microscale. This problem associated with the parallel microfluidic system may be adapted for use in the system according to the present invention. The bifurcating regions in the channel may be configured to convey a fluid through one channel after the bifurcating region. For example, in FIG. 3 the bifurcating region where 1^{st} sample delivery channel meets the reaction zone channel may be configured to enable the sample conveyed through the 1st sample delivery channel to flow towards the 1^{st} reaction zone and not flow in the opposite direction to the 2^{nd} reaction zone. The configuration of the bifurcating regions may be altered so that the fluid may be directed down either channel after the bifurcating region.

In a preferred embodiment, the microfluidic system according to the present invention comprises one or more valves to control the flow of fluids through the microchannels. The microfluidic system may comprise one or more valves selected from a reaction zone valve (12), first sample delivery channel valve (13), second sample delivery channel valve (14), reagent delivery channel valve (15), reagent delivery channel valve (19) positioned after the bifurcating region, waste channel valve (20), further waste channel valve (17) and fluid delivery channel valve (22).

In one embodiment, the microfluidic system controls flow of the fluids through the channels by a combination of valve control and configuration of bifurcating regions.

The principle parts of the microfluidic system are shown in FIG. 3. The system comprises a 1^{st} reaction zone (1), a 2^{nd} reaction zone (2), a reagent delivery channel (3) configured to deliver one or more reagents to the 1^{st} reaction zone (1), a waste channel (7) to remove waste from the 2^{nd} reaction zone (2), a 1^{st} sample delivery channel (5) configured to deliver a sample to the 1^{st} reaction zone (1) and a 2^{nd} sample delivery channel (6) configured to deliver a sample to the 2^{nd} reaction zone (2); wherein each reaction zone comprises means for retaining one or more reagents; and wherein the 1^{st} reaction zone (1) and 2^{nd} reaction zone (2) are connected in series by a reaction zone channel (4).

The reaction zone channel connects the 1^{st} reaction zone and 2^{nd} reaction zone in series and thereby allows the solution comprising the one or more reagents to flow from the 1^{st} reaction zone to the 2^{nd} reaction zone. Therefore, the same solution comprising one or more reagents is delivered to both reaction zones. This arrangement of the channels in the system according to the present invention, wherein the 1^{st} reaction zone and 2^{nd} reaction zone are connected in series by the reaction zone channel is advantageous because it ensures that the samples entered into each reaction zone are subjected to the same reagent conditions and provides more accurate and reliable results.

In a preferred embodiment the reagent delivery channel and the reaction zone channel (positioned between the 1^{st} reaction zone and 2^{nd} reaction zone) are configured to deliver the same volume of the one or more reagents to each reaction zone. This ensures that the same volume of the solution comprising the reagents is conveyed through each reaction zone, thereby allowing the same quantity of one or more reagents to be retained in each reaction zone. This embodiment is typically achieved by ensuring that the reagent delivery channel and the reaction zone channel have a length and diameter which coveys the same volume of fluid to the 1^{st} and 2^{nd} reaction zones. For example, the reagent delivery channel and the reaction zone channel may be the same length and diameter.

An example of this embodiment is shown in FIG. 13 where the region of the reagent delivery channel between the bifurcating region and the further valve 19 in the reagent delivery channel is region 33, the region of the reagent delivery channel between valve 19 and the 1^{st} reaction zone is region 34, the region of the reaction zone channel between the 1^{st} reaction zone and the reaction zone valve 12 is region 35, the region of the reaction zone channel between the reaction zone valve 12 and the 2^{nd} reaction zone is region 26 and the region of the waste channel from the 2^{nd} reaction zone to the valve 20 in the waste channel is region 37.

In a preferred embodiment, the total volume of fluid required to fill regions 33 and 34 is the same as the total volume of fluid required to fill regions 35 and 36. This embodiment is advantageous because if washing steps are carried out it ensures that the same quantity of reagents remaining in the channels is conveyed through each reaction zone. In this embodiment, if valves are present in the system the exact positioning of valves 19 and 12 is not particularly important provided that the total volume of fluid required to fill regions 33 and 34 is the same as regions 35 and 36.

In a further preferred embodiment the total volume of fluid required to fill regions 34 and 35 is the same as the total volume of fluid required to fill regions 36 and 37. This embodiment is particularly advantageous because it ensures that the same volume of solution comprising one or more reagents is present in the microfluidic channel each side of the reaction zones is the same. For example, step b) may be carried out when valves 19 are 20 are closed, and preferably valve 12 is closed, and therefore the same quantity of reagents is present in the channels either side of the reaction zones.

In one preferred embodiment each of regions 33, 34, 35, 36 and 37 of the channels are configured to deliver the same volume of fluid. Each of regions 33, 34, 35, 36 and 37 may be the same length and diameter.

In a preferred embodiment, the reaction zone channel connecting the 1^{st} reaction zone and 2^{nd} reaction zone comprises a reaction zone valve. The reaction zone valve may be opened to allow solutions comprising reagents to be passed through both the 1^{st} reaction zone and the 2^{nd} reaction zone. The reaction zone valve may be closed after the reagents have been passed through the 1^{st} and 2^{nd} reaction zone and one or more reagents are retained in the reaction zone. When the reaction zone valve is closed, separate samples may then be conveyed through each reaction zone without coming into contact. In the assay method according to the present invention, steps a) and b) are preferably carried out when the reaction zone valve is open and the reaction zone valve is closed before step c).

In one embodiment, one or more valves are positioned in the system to ensure that the same volume of one or more reagents is delivered to each reaction zone. For example, in one embodiment of the microfluidic system shown in FIG. 13, the valve 19 in the reagent delivery channel and the valve 12 in the reaction zone channel are positioned to ensure that the volume of fluid required to fill regions 33 and 34 is the same as the volume required to fill regions 35 and 36. In a further preferred embodiment, valve 19 in the reagent delivery channel, the valve 12 in the reaction zone channel and the valve 20 in the waste channel from the 2^{nd} reaction zone are positioned to ensure that the volume of fluid required to fill regions 34 and 35 is the same as the volume of fluid required to fill regions 36 and 37. In one embodiment the valve 19 in the reagent delivery channel, the valve 12 in the reaction zone channel and the valve 20 in the waste channel from the 2^{nd} reaction zone are positioned to ensure that the volume of fluid required to fill each of regions 33, 34, 35, 36 and 37 in the microfluidic channels are the same.

In a further preferred embodiment the 1^{st} sample delivery channel and the 2^{nd} sample delivery channel are configured to deliver the same volume of the samples to each reaction zone. This may be achieved by ensuring that both the sample delivery channels have a length and diameter which coveys the same volume of fluid to the 1^{st} and 2^{nd} reaction zones. The sample delivery channels may be directly connected to the reaction zones. Alternatively, the sample delivery channels converge with the reaction zone channel, as shown for example in FIG. 4. In this embodiment both the sample delivery channels and the reaction zone channel are preferably configured to deliver the same quantity of the samples to each reaction zone.

In the embodiment wherein each sample delivery channel comprises a valve (valves 13 and 14 shown in FIG. 4) the valves 13 and 14 are preferably positioned in the sample delivery channels to ensure that the same volume of each sample is delivered to each reaction zone.

In one embodiment of the present invention, the system is configured to allow removal of waste sample from the 1^{st} reaction zone via the reagent delivery channel. This embodiment is shown in FIGS. 3, 4 and 8. In FIG. 8 arrow 26 shows the flow direction of the sample through the 1^{st} sample delivery channel to the 1^{st} reaction zone and the flow of waste sample out through the reagent delivery channel.

In the assay method according to the present invention wherein the system is configured to allow removal of waste sample from the 1^{st} reaction zone via the reagent delivery channel, step c) comprises removing waste sample from the 1^{st} reaction zone via the reagent delivery channel and removing waste sample from the 2^{nd} reaction zone via the waste channel.

The system preferably comprises one or more valves, wherein the valves are positioned to allow: the one or more reagents to be delivered to the 1^{st} reaction zone and the 2^{nd} reaction zone via the reaction zone channel; a sample to be delivered to the 1^{st} reaction zone from the 1^{st} sample delivery channel without delivery to the 2^{nd} reaction zone; and a sample to be delivered to the 2^{nd} reaction zone from the 2^{nd} sample delivery channel without delivery to the 1^{st} reaction zone. In this embodiment, the system preferably comprises a valve positioned in the reaction zone channel, a valve positioned in the 1^{st} sample delivery channel and a valve positioned in the 2^{nd} sample delivery channel.

FIG. 4 shows an example configuration of the valves in the system, where valve 12 is positioned in the reaction zone channel and valves 13 and 14 are positioned in the 1^{st} sample delivery channel and 2^{nd} sample delivery channel respectively. In step a) of the method according to the present invention, valve 12 is open and valves 13 and 14 closed to allow the solution comprising one or more reagents to be delivered to the 1^{st} reaction zone and 2^{nd} reaction zone. In step c), valve 13 is open and valve 12 is closed to allow a sample to be delivered to the 1^{st} reaction zone from the 1^{st} sample delivery channel without delivery to the 2^{nd} reaction zone, and removal of waste sample from the 1^{st} reaction zone via the reagent delivery channel; and valve 14 is open and valve 12 is closed to allow a sample to be delivered to the 2^{nd} reaction zone from the 2^{nd} sample delivery channel without delivery to the 1^{st} reaction zone, and removal of waste sample from the 2^{nd} reaction zone via the waste channel.

In a further embodiment of the present invention the system comprises a further waste channel to remove waste from the 1^{st} reaction zone.

In the assay method according to the present invention, wherein the system comprises a further waste channel to remove waste from the 1^{st} reaction zone, step c) preferably comprises removing waste sample from the 1^{st} reaction zone via the further waste channel and removing waste sample from the 2^{nd} reaction zone via the waste channel.

An example configuration of the system comprising a further waste channel is shown in FIG. 5, where 16 is the further waste channel and 17 is a valve in the further waste channel. FIG. 9 shows the system of FIG. 5, where arrow 24 represents the flow of the one or more reagents through the reagent delivery channel into the 1^{st} reaction zone, through the reaction zone channel into the 2^{nd} reaction zone and through the waste channel. In FIG. 9 25 represents the flow of the sample through the 2^{nd} sample delivery channel to the 2^{nd} reaction zone and the flow of waste sample through the waste channel. In FIG. 9 27 shows the flow of the sample through the 1^{st} sample delivery channel to the 1^{st} reaction zone and the flow of waste sample through the further waste channel.

In this embodiment, the system preferably comprises one or more valves, wherein the valves are positioned to allow: the one or more reagents to be delivered to the 1^{st} reaction zone and the 2^{nd} reaction zone via the reaction zone channel without delivery to the further waste channel; and removal of waste sample from the 1^{st} reaction zone via the further waste channel without delivery to the reagent delivery channel. Preferably the reagent delivery channel comprises a valve and the further waste channel comprises a valve.

FIG. 5 shows an example configuration of the valves in the system where valve 12 is positioned in the reaction zone channel, valves 13 and 14 are positioned in the 1^{st} sample delivery channel and 2^{nd} sample delivery channel respectively, valve 15 is positioned in the reagent delivery channel and valve 17 is positioned in the further waste channel. In step a) according to the method of the present invention, valves 15 and 12 are open and valves 17, 13 and 14 are closed to allow the one or more reagents to be delivered to the 1^{st} reaction zone and 2^{nd} reaction zone via the reaction zone channel and without delivery to the further waste channel. In step c) valves 13 and 17 are open and valves 12 and 15 are closed to allow a sample to be delivered to the 1^{st} reaction zone from the 1^{st} sample delivery channel without delivery to the 2^{nd} reaction zone, and removal of waste sample from the 1^{st} reaction zone via the further waste channel without delivery to the reagent delivery channel; and valve 14 is open and valve 12 is closed to allow a sample to be delivered to the 2^{nd} reaction zone from the 2^{nd} sample delivery channel without delivery to the 1^{st} reaction zone, and removal of waste sample from the 2^{nd} reaction zone via the waste channel.

FIG. 6 shows an example configuration of the valves in the system, where valves 15, 19, 20 and 12 are open and valves 17, 13 and 14 are closed to allow the one or more reagents to be delivered to the 1^{st} reaction zone and 2^{nd} reaction zone via the reaction zone channel without delivery to the further waste channel (in step a according to the method of the present invention). Valves 13 and 17 are open and valves 12 and 15 and/or 19 are closed to allow a sample to be delivered to the 1^{st} reaction zone from the 1^{st} sample delivery channel without delivery to the 2^{nd} reaction zone, and removal of waste sample from the 1^{st} reaction zone via the further waste channel without delivery to the reagent delivery channel; valves 14 and 20 are open and valve 12 is closed to allow a sample to be delivered to the 2^{nd} reaction zone from the 2^{nd} sample delivery channel without delivery to the 1^{st} reaction zone, and removal of waste sample from the 2^{nd} reaction zone via the waste channel (in step c according to the method of the present invention).

In one embodiment, the further waste channel is positioned to allow a liquid to be conveyed through the sample delivery channel to the further waste channel without passing through the reaction zones, as shown for example in FIG. 5. In this embodiment the method according to the present invention preferably comprises a further step after step a) and before step c) of flushing a liquid through the reagent delivery channel via the further waste channel without passing through the reaction zones. This step may be carried out before, during or after step b) of the method and is preferably carried out before step b).

In this embodiment, the system preferably comprises one or more valves which allow a liquid to be flushed through the sample delivery channel to the further waste channel without passing through the reaction zones, as shown for example by flow arrow 28 in FIG. 10. Typically, valves 22 and 17 will be open and valve 19 closed to allow this flow (28) of liquid through the system. The flow of fluid shown by arrow 28 may be conveyed through the system after the flow of reagents shown by arrow 24. The flow of fluid 28 preferably removes the reagents, such as beads, from the reagent delivery channel 3.

The assay method according to the present invention may also comprise a further step after step a) and before step c) of flushing a liquid through the reagent delivery channel, the 1^{st} reaction zone, the 2^{nd} reaction zone and the waste channel from the 2^{nd} reaction zone. This step may be carried out during or after step b) of the method. The flow of liquid in this step is the same as the flow shown by arrow 24 in FIGS. 8-10. In this embodiment, wherein the microfluidic system comprises valves, the reagent delivery channel valves 15 and 19 are open, the valve 20 in the waste channel from the 2^{nd} reaction zone is open and the samples delivery channel valves 13 and 14 are closed. In the embodiment, wherein the system comprises a further waste channel comprising a valve 17, this valve is closed in this step. The liquid may enter into the reagent delivery channel from a delivery channel, as shown for example as channel 21 in FIG. 7. If the liquid does enter from delivery channel 21, valve 22 would be open and valve 15 closed.

This further step of flushing a liquid through the reagent delivery channel, the 1^{st} reaction zone, the 2^{nd} reaction zone and the waste channel from the 2^{nd} reaction zone is preferably carried out after the step of flushing a liquid through the reagent delivery channel via the further waste channel without passing through the reaction zones. The combination of both these steps is advantageous because it provides further control over the quantity of reagents which are passed through the reaction zones. Referring to FIG. 10, after the solution comprising one or more reagents has been conveyed through the 1^{st} reaction zone and the 2^{nd} reaction zone in step a) (arrow 24), a liquid is flushed through the reagent delivery channel via the further waste channel without passing through the reaction zones (arrow 28) to remove the reagents from this part of the microfluidic system. At this stage, the solution comprising the one or more reagents only remains in the region of the reagent delivery channel between the bifurcating region and the 1^{st} reaction zone (33 and 34 of FIG. 13), the reaction zone channel (35 and 36 of FIG. 13) and the waste channel from the 2^{nd} reaction zone (37 of FIG. 13 and the region after valve 20). Subsequently, the step of flushing a liquid through the reagent delivery channel, the 1^{st} reaction zone, the 2^{nd} reaction zone and the waste channel pushes through remaining solution comprising one or more reagents (conveyed in step a)) thus causing the reagents present in the reagent delivery channel (regions 33 and 34) to pass through the 1^{st} reaction zone and the reagents present in the reaction zone channel (regions 35 and 36) to pass through the 2^{nd} reaction zone.

In the preferred embodiment where the total volume of fluid required to fill regions 33 and 34 is the same as the total volume of fluid required to fill regions 35 and 36, the same volume of the one or more reagents is conveyed through each reaction zone when this step is carried out. Further, if step b) of retaining one or more reagents in the reaction zones is carried out during or after the step of flushing a liquid through the reagent delivery channel, the 1^{st} reaction zone, the 2^{nd} reaction zone, the configuration of regions 33, 34, 35 and 36 will ensure that the same quantity of the one or more reagents is retained in each reaction zone. This ensures that the conditions in both reaction zones are the same and provides more accurate assay results. This advantage is also achieved in the preferred embodiment where regions 33, 34, 35, 36 and 37 of the channels are configured to deliver the same volume of the one or more reagents.

The liquid flushed through the reagent delivery channel, the 1^{st} reaction zone, the 2^{nd} reaction zone and the waste channel may be any suitable liquid, for example a wash solution. The solution flushed through in this step acts to push through any remaining solution comprising the one or more reagents conveyed through the reagent delivery channel, the 1^{st} and 2^{nd} reaction zones and the waste channel in step a).

In the assay method according to the present invention the method preferably comprises a further step before step a) of flushing a liquid, such as a wash buffer, through the reagent delivery channel, the 1^{st} reaction zone, the 2^{nd} reaction zone and waste channel.

In a further embodiment of the present invention the microfluidic system comprises a reaction zone waste channel to remove waste from the 1^{st} reaction zone. An example of this embodiment is shown in FIG. 11 where the reaction zone waste channel 29 is shown, 30 is a valve in the reaction zone waste channel and 31 is an outlet from the reaction zone waste channel. This reaction zone waste channel allows a fluid to be passed through the 1^{st} reaction zone and to the waste channel without passing through the 2^{nd} reaction zone. This is particularly important when it is required to separately wash the reaction zones before conveying one or more further reagents through the reaction zones. Accordingly, the method according to the present invention may also comprise a further step of flushing a liquid through the reagent delivery channel, the 1^{st} reaction zone and the reaction zone waste channel from the reaction zone channel. This step is preferably carried out after step c) of conveying the samples through the reaction zones. This step ensures that any remaining sample which was conveyed to the 1^{st} reaction zone in step c) still present in the reagent delivery channel and/or the reaction zone channel is removed from the microfluidic system without passing through the 2^{nd} reaction zone.

The flow of liquid in this step is shown by arrow 32 in FIG. 12. In this embodiment, wherein the microfluidic system comprises valves, the reagent delivery channel valve 19 is open and the valve 30 in the reaction zone waste channel is open and the sample delivery channel valve 13 and reaction zone channel valve 12 are closed. In the embodiment where the reagent delivery channel comprises a waste channel 16, the valve 17 is also closed. Either the reagent delivery channel valve 5 is open or if the liquid enters from a delivery channel 21, as shown in FIG. 12, valve 22 would be open and valve 15 closed.

The liquid flushed through the reagent delivery channel, the 1^{st} reaction zone and the reaction zone waste channel may be any suitable liquid, for example a wash solution.

The method according to the present invention may also comprise a step of flushing a liquid through the reagent delivery channel, the 1^{st} reaction zone, the 2^{nd} reaction zone and the further waste channel from the 2^{nd} reaction zone. The flow of liquid in this step is shown by arrow 24 in FIGS. 8-10. If the system comprises valves, the same valves are open and closed as discussed above for the flow of liquid shown by arrow 24. This step is preferably carried out after step c) of conveying the samples through the reaction zones. This step is also preferably carried out after the step of flushing a liquid through the reagent delivery channel, the 1^{st} reaction zone and the reaction zone waste channel from the reaction zone channel. In this embodiment, the step of flushing a liquid through the reagent delivery channel, the 1^{st} reaction zone and the reaction zone waste channel from the reaction zone channel preferably removes remaining sample which was conveyed to the 1^{st} reaction zone without passing through the 2^{nd} reaction zone. The subsequent step of flushing a liquid through the reagent delivery channel, the 1^{st} reaction zone, the 2^{nd} reaction zone and the further waste channel from the 2^{nd} reaction zone then may remove remaining sample which was conveyed to the 2^{nd} reaction zone without passing through the 1^{st} reaction zone. This is advantageous because it ensures that there is no cross-contamination of the samples in the reaction zones.

The liquid flushed through the reagent delivery channel, the 1^{st} reaction zone, 2^{nd} reaction zone and the waste channel from the 2^{nd} reaction zone may be any suitable liquid, for example a wash solution.

The method according to the present invention may also comprise a further step of conveying one or more further reagents into the 1^{st} reaction zone and the 2^{nd} reaction zone. The flow of solution in this step is shown by arrow 24 is FIGS. 8-10. If the system comprises valves, the same valves are open and closed as discussed above for the flow of liquid shown by arrow 24. This step is preferably carried out after step c) and after the steps of flushing a liquid through the reagent delivery channel, the 1^{st} reaction zone and the reaction zone waste channel and flushing a liquid through the reagent delivery channel, the 1^{st} reaction zone, the 2^{nd} reaction zone and the further waste channel from the 2^{nd} reaction zone. The one or more further reagents conveyed through the 1^{st} reaction zone and 2^{nd} reaction zone may be any suitable reagent as described above. For example, a solution comprising antibodies, which may be labeled, specific for a target antigen may be conveyed through the 1^{st} reaction zone and 2^{nd} reaction zone.

The method according to the present invention may comprise one or more further steps of flushing a liquid through the reaction zones to wash the reaction zones and introduce further reagents. Preferably the method comprises repeating the step of flushing a liquid through the reagent delivery channel, the 1^{st} reaction zone and the reaction zone waste channel from the reaction zone channel, flushing a liquid through the reagent delivery channel, the 1^{st} reaction zone, the 2^{nd} reaction zone and the further waste channel from the 2^{nd} reaction zone; and conveying one or more further reagents into the 1^{st} reaction zone and the 2^{nd} reaction zone. These steps may be repeated to allow the 1^{st} reaction zone and 2^{nd} reaction zone to be washed before one or more further reagents are conveyed through the reaction zones. For example, after a solution comprising antibodies has been conveyed through the reaction zones, as discussed above, the reaction zones may be washed and then a substrate, such as—methylumbelliferyl phosphate (MUP) and fluorescein diphosphate (FDP), may be conveyed through the reaction zones.

After the final reagents have been conveyed through the reaction zones, the method may comprise a final step of washing the reaction zones. This final washing step is preferably carried out by flushing a liquid through the reagent delivery channel, the 1^{st} reaction zone and the reaction zone waste channel from the reaction zone channel and flushing a liquid through the reagent delivery channel, the 1^{st} reaction zone, the 2^{nd} reaction zone and the further waste channel from the 2^{nd} reaction zone.

The microfluidic system according to the present invention may also comprise one or more further reaction zones. Each further reaction zone comprises means for retaining one or more reagents, as discussed above for the 1^{st} and 2^{nd} reaction zones. In this embodiment, each further reaction zone is connected to a sample delivery channel configured to deliver a sample to the reaction zone. Depending upon the required use of the system, one or more of the further reaction zones may be connected to the same sample delivery channel. Alternatively, each further reaction zone is independently connected to a separate sample delivery channel to prevent cross-contamination of the samples. Each further reaction zone may also be connected to a waste channel to allow waste sample from the reaction zone to be removed via the waste channel.

In this embodiment, the one or more further reaction zones are connected in series by one or more further reaction zone channels to the 1^{st} reaction zone and the 2^{nd} reaction zone. Accordingly, one or more further samples, which may be further test samples and/or calibration samples, can be delivered to the reaction zones and subjected to the same reaction conditions as the 1^{st} and 2^{nd} reaction zones. This embodiment allows a plurality of samples to be analysed simultaneously under the same reaction conditions.

The assay method according to the present invention may also comprise delivery of the one or more reagents to these further reaction zones via the further reaction zone channels and delivery of the further samples to each reaction zone via each further sample delivery channel. The method may also comprise removal of waste sample from each further waste channel via further waste channels.

The present invention also provides a cartridge system comprising:
(a) a reagent component for storing one or more reagents; and
(b) a processing component for processing the one or more reagents in an assay,
   wherein the processing component comprises a microfluidic system as defined above;
   wherein the reagent component and the processing component are configured to be coupled together to form a cartridge.

The cartridge system preferably further comprising a sensing component comprising at least one sensing element for detecting an analyte. The cartridge system also preferably comprises a sample preparation component for preparing a sample for the assay.

The cartridge system is preferably the cartridge system disclosed in PCT/GB2007/003666 (the contents of which are incorporated by reference), wherein the processing component comprises a microfluidic system according to the present invention.

In the assay method according to the present invention, the microfluidic system may be in a cartridge system, as defined above. In this embodiment after step c) the method further comprises the step of coupling the cartridge system to an assay device configured to accept the cartridge, wherein step (d) is carried out using the assay device.

The present invention also provides a microfluidic system comprising a 1^{st} reaction zone, a 2^{nd} reaction zone, a reagent delivery channel configured to deliver one or more reagents to the 1^{st} reaction zone, a waste channel to remove waste from the 2^{nd} reaction zone, a 1^{st} sample delivery channel configured to deliver a sample to the 1^{st} reaction zone, a 2^{nd} sample delivery channel configured to deliver a sample to the 2^{nd} reaction zone and a means for retaining one or more reagents in the 1^{st} reaction zone and 2^{nd} reaction zone; wherein the means for retaining one or more reagents is positioned so that it retains the same quantity of one or more reagents in the 1^{st} reaction zone and the 2^{nd} reaction zone.

The means for retaining one or more reagents are positioned outside of the reaction zones but is positioned, preferably in close proximity and at an equal distance between the reaction zones, so that it affects the retention of reagents in each reaction zone equally. Preferably the means for retaining one or more reagents is magnetic or magnetisable and more preferably is a magnet positioned between the 1^{st} reaction zone and the 2^{nd} reaction zone, as shown for example in FIG. 14. This is particularly advantageous because it ensures that both reaction zones are subjected to the same magnetic field strength and, therefore, the same quantity of the reagents is retained in each reaction zone.

The 1^{st} reaction zone, the 2^{nd} reaction zone, the reagent delivery channel configured to deliver one or more reagents to the 1^{st} reaction zone, the waste channel to remove waste from the 2^{nd} reaction zone, the 1^{st} sample delivery channel configured to deliver a sample to the 1^{st} reaction zone and the 2^{nd} sample delivery channel configured to deliver a sample to the 2^{nd} reaction zone are preferably as described above.

1. A microfluidic system comprising a 1^{st} reaction zone, a 2^{nd} reaction zone, a reagent delivery channel configured to deliver one or more reagents to the 1^{st} reaction zone, a waste channel to remove waste from the 2^{nd} reaction zone, a 1^{st} sample delivery channel configured to deliver a sample to the 1^{st} reaction zone and a 2^{nd} sample delivery channel configured to deliver a sample to the 2^{nd} reaction zone; wherein the microfluidic system comprises a means for retaining one or more reagents in each reaction zone; and wherein the 1^{st} reaction zone and 2^{nd} reaction zone are connected in series by a reaction zone channel.

2. A microfluidic system according to claim 1, wherein system is configured to allow removal of waste sample from the 1^{st} reaction zone via the reagent delivery channel.

3. A microfluidic system according to claim 1, wherein the system comprises one or more valves, wherein the valves are positioned to allow: the one or more reagents to be delivered to the 1^{st} reaction zone and the 2^{nd} reaction zone via the reaction zone channel; a sample to be delivered to the 1^{st} reaction zone from the 1^{st} sample delivery channel without delivery to the 2^{nd} reaction zone; and a sample to be delivered to the 2^{nd} reaction zone from the 2^{nd} sample delivery channel without delivery to the 1^{st} reaction zone.

4. A microfluidic system according to claim 3, wherein the reaction zone channel comprises a valve, the 1^{st} sample delivery channel comprises a valve and the 2^{nd} sample delivery channel comprises a valve.

5. A microfluidic system according to claim 1, wherein the system comprises a further waste channel to remove waste from the 1^{st} reaction zone.

6. A microfluidic system according to claim 5, wherein the system comprises one or more valves, wherein the valves are positioned to allow: the one or more reagents to be delivered to the 1^{st} reaction zone and the 2^{nd} reaction zone via the reaction zone channel without delivery to the further waste channel; and removal of waste sample from the 1^{st} reaction zone via the further waste channel without delivery to the reagent delivery channel.

7. A microfluidic system according to claim 6, wherein the reagent delivery channel comprises a valve and the further waste channel comprises a valve.

8. A microfluidic system according to claim 5, wherein the further waste channel is positioned to allow a liquid to be conveyed through the sample delivery channel to the further waste channel without passing through the reaction zones.

9. A microfluidic system according to claim 1, wherein the reagent delivery channel and the reaction zone channel are configured to deliver the same volume of the one or more reagents to each reaction zone.

10. A microfluidic system according to claim 1, wherein the means for retaining one or more reagents is magnetic or magnetisable.

11. A microfluidic system according to claim 10, wherein the means for retaining one or more reagents is a magnet positioned between the 1^{st} reaction zone and the 2^{nd} reaction zone.

12. A microfluidic system according to claim 1, wherein the system comprises one or more further reaction zones, wherein each further reaction zone is connected to a sample delivery channel configured to deliver a sample to the reaction zone, wherein the one or more further reaction zones are connected in series by one or more further reaction zone channels to the 1^{st} reaction zone and the 2^{nd} reaction zone, and wherein each further reaction zone comprises a means for retaining one or more reagents.

13. A microfluidic system according to claim 1, wherein the system comprises a reaction zone waste channel to remove waste from the 1^{st} reaction zone via the reaction zone channel.

14. A cartridge system comprising:
(a) a reagent component for storing one or more reagents; and
(b) a processing component for processing the one or more reagents in an assay, wherein the processing component comprises a microfluidic system according to claim 1;
wherein the reagent component and the processing component are configured to be coupled together to form a cartridge.

15. A cartridge system according to claim 14, further comprising a sensing component comprising at least one sensing element for detecting an analyte.

16. A cartridge system according to claim 14, further comprising a sample preparation component for preparing a sample for the assay.

17. (canceled)

18. An assay method for one or more analytes in a sample, which method comprises:
a) conveying a solution comprising one or more reagents through a microfluidic system of claim 1, wherein the solution comprising one or more reagents is conveyed into the 1^{st} reaction zone and the 2^{nd} reaction zone;
b) retaining one or more reagents in the 1^{st} reaction zone and 2^{nd} reaction zone;
c) conveying a sample through the 1^{st} sample delivery channel into the 1^{st} reaction zone and conveying a sample through the 2^{nd} sample delivery channel into the 2^{nd} reaction zone; and
d) assaying for the one or more analytes.

19. An assay method according to claim 18, wherein the sample conveyed into the 1^{st} reaction zone is a test sample and the sample conveyed into the 2^{nd} reaction zone is a calibration sample comprising a control liquid and the method comprises a step of calibrating the assay results of the 1^{st} reaction zone with the assay results of the 2^{nd} reaction zone.

20. An assay method according to claim 18, wherein the sample conveyed into the 1^{st} reaction zone is a test sample and the sample conveyed into the 2^{nd} reaction zone is a calibration sample comprising one or more aliquots of an analyte, each aliquot having a different known quantity of the one or more analytes, and the method comprises a step of calibrating the quantity of the one or more analytes in the test sample against the known and determined quantity of the one or more analytes in the calibration sample.

21. An assay method according to claim 18, wherein in step b) the means for retaining one or more reagents are applied to the 1^{st} reaction zone and 2^{nd} reaction zone after the solution comprising the one or more reagents has filled the 1^{st} reaction zone and the 2^{nd} reaction zone.

22. An assay method according to claim 18, wherein step c) comprises removing waste sample from the 1^{st} reaction zone via the reagent delivery channel and removing waste sample from the 2^{nd} reaction zone via the waste channel.

23. An assay method according to claim 18, wherein step c) comprises removing waste sample from the 1^{st} reaction zone via the further waste channel and removing waste sample from the 2^{nd} reaction zone via the waste channel.

24. An assay method according to claim 18, comprising a further step after step a) and before step c) of flushing a liquid through the reagent delivery channel to the further waste channel without it passing through the reaction zones.

25. An assay method according to claim 18, wherein the method comprises a further step before step a) of flushing a liquid through the reagent delivery channel, the 1^{st} reaction zone, the 2^{nd} reaction zone and waste channel.

26. An assay method according to claim 18, wherein the method comprises a further step after step a) and before step c) of flushing a liquid through the reagent delivery channel, the 1^{st} reaction zone, the 2^{nd} reaction zone and the waste channel.

27. An assay method according to claim 18, wherein the method comprises a further step after step c) of flushing a liquid through the reagent delivery channel, the 1^{st} reaction zone and the reaction zone waste channel.

28. An assay method according to claim 18, wherein the method comprises a further step after step c) of flushing a liquid through the reagent delivery channel, the 1^{st} reaction zone, the 2^{nd} reaction zone and the further waste channel from the 2^{nd} reaction zone.

29. An assay method according to claim 18, wherein the method comprises a further step after step c) of conveying a solution comprising one or more reagents into the 1^{st} reaction zone and the 2^{nd} reaction zone.

30. An assay method according to claim 18, wherein the one or more reagents retained in step b) are magnetic or magnetisable reagents and/or are each attached to a magnetic or magnetisable substance.

31. An assay method according to claim 30, wherein the one or more reagents are magnetic beads and/or the magnetic or magnetisable substance is a magnetic bead.

32. An assay method according to claim 18, wherein the microfluidic system is in a cartridge system, wherein after step c) the method further comprises the step of coupling the cartridge system to an assay device configured to accept the cartridge, wherein step (d) is carried out using the assay device.

33. A microfluidic system comprising a 1^{st} reaction zone, a 2^{nd} reaction zone, a reagent delivery channel configured to deliver one or more reagents to the 1^{st} reaction zone, a waste channel to remove waste from the 2^{nd} reaction zone, a 1^{st} sample delivery channel configured to deliver a sample to the 1^{st} reaction zone, a 2^{nd} sample delivery channel configured to deliver a sample to the 2^{nd} reaction zone and a means for retaining one or more reagents in the 1^{st} reaction zone and 2^{nd} reaction zone; wherein the means for retaining one or more reagents is positioned so that it retains the same quantity of one or more reagents in the 1^{st} reaction zone and the 2^{nd} reaction zone.

34. A microfluidic system according to claim 33, wherein the means for retaining one or more reagents is magnetic or magnetisable.

35. A microfluidic system according to claim 34, wherein the means for retaining one or more reagents is a magnet positioned between the 1^{st} reaction zone and the 2^{nd} reaction zone.
